(19) United States
(12) Patent Application Publication
(10) Pub. No.: US 2004/0250814 A1
(43) Pub. Date: Dec. 16, 2004

(54) Portable anesthesia apparatus

(75) Inventors: Terry Post, Bellmont, NY (US); George Dickherber, Wentzville, MO (US)

Correspondence Address: JOHN W KEPLER, III, 7733 FORSYTH BLVD., 12TH FLOOR, ST LOUIS, MO, 63105, US

(73) Assignee: Nonlinear Medical Systems, Inc., Wentzville, MO

(21) Appl. No.: 10/458,594

(22) Filed: Jun. 10, 2003

(51) Int. Cl. A61M 15/00

(52) U.S. Cl. 128/203.12

(57) ABSTRACT

The present invention is directed to a portable anesthesia apparatus that is highly accurate and durable. The portable anesthesia apparatus may contain one or more different liquid anesthesia agents. The portable anesthesia apparatus contains a reservoir defined by walls for containing a liquid anesthetic agent, a control shaft having a tapered portion, a seat having a tapered receiving portion, the seat having an exit port, wherein the tapered receiving portion of the seat receives the tapered portion of the control shaft, wherein the exit port is below the tapered receiving portion of the seat, wherein an evaporation chamber containing a conductive material is below the exit port, and wherein rotating the control shaft releases liquid anesthetic agent onto the conductive material for evaporation into a carrier gas stream.

### FIELD OF THE INVENTION

The present invention relates to a portable anesthesia apparatus used for administering general anesthetics to human and animal patients.

### BACKGROUND OF THE INVENTION

Most anesthesia machines are intended to remain in the secure environment of a hospital, medical office, or veterinary office and require electrical supply and/or carrier gas supply from a dedicated source. Often, medical practitioners desire the ability to conveniently transport the anesthesia machine to a patient in a remote location that lacks dedicated electric or gas supplies. Such remote locations may include farms or even a battlefield.

Moreover, most anesthesia machines are relatively fragile, requiring delicate care during transport to remote locations. Of course, the transport of most anesthesia machines to a location for a single use is not economically feasible.

The performance of most anesthesia machines is subject to changes in ambient conditions, such as temperature, pressure and humidity. Many prior anesthesia machines fail to operate effectively in ambient conditions other than those found in the stable setting of the medical practitioner's facility.

What is desired is a portable, durable anesthesia apparatus that may be transported to patients in remote locations and operate effectively under a wide variety of temperature, pressure and humidity conditions.

### SUMMARY OF THE INVENTION

The present invention provides a portable anesthesia apparatus, including:

one or more reservoirs containing a liquid anesthetic agent, a rotatable control shaft with a tapered portion, a seat for receiving the tapered portion of the control shaft, the seat having an exit port, and whereby rotating the control shaft releases the liquid anesthetic agent in a drop wise fashion from the exit port into an evaporation chamber containing a conductive material, and whereby the liquid anesthetic agent is evaporated into a carrier gas which is administered to a patient.

It is an aspect of the present invention to provide a portable anesthetic apparatus.

It is another aspect of the present invention to provide an anesthetic apparatus that is durable.

It is another aspect of the present invention to provide an anesthetic apparatus capable of operating in a wide variety of ambient conditions.

It is another aspect of the present invention to provide an anesthetic apparatus capable of containing and/or simultaneously applying one or more different liquid anesthetic agents.

It is another aspect of the present invention to provide a rotatable control shaft with a tapered portion that is integral to a seat containing an exit port for the release of a liquid anesthetic agent from the exit port in a drop wise fashion.

It is another aspect of the present invention to provide a portable anesthetic apparatus that can be made from one piece of metal stock.

It is another aspect of the present invention to provide an anesthetic apparatus which may be operated without external electrical sources or dedicated gas supplies.

These and other aspects of the present invention are achieved herein.

### BRIEF DESCRIPTION OF THE DRAWINGS

The present invention is illustrated by the embodiments shown in the following drawings:

FIG. 1 is a schematic representation of a cylinder of the portable anesthetic apparatus.

FIG. 2 is a schematic representation of a seat of the cylinder.

FIG. 3 is a schematic representation of the seat of the cylinder along with a mesh sheet of the present invention.

FIG. 4 is a schematic representation of the cylinder and the fill port.

FIG. 5 is a schematic representation of the fill reservoir.

FIG. 6 is a schematic representation of a three chambered portable anesthetic apparatus.

FIG. 7 is a schematic representation of a universal connector.

FIG. 8 is a schematic representation of a control shaft.

FIG. 9 is a schematic representation of the portable anesthesia apparatus with an extension for flexible fluid container.

FIG. 10 is a top-down view of the three chambered portable anesthetic apparatus.

FIG. 11 is a side-view of the three chambered portable anesthetic apparatus.

FIG. 12 is a top-down view of the three chambered portable anesthetic apparatus with a top cap removed.

FIG. 13 is a bottom-view of the three chambered portable anesthetic apparatus with a bottom cap removed.

FIG. 14 is a top-down view of the three chambered portable anesthetic apparatus.

### DETAILED DESCRIPTION OF THE PREFERRED EMBODIMENTS

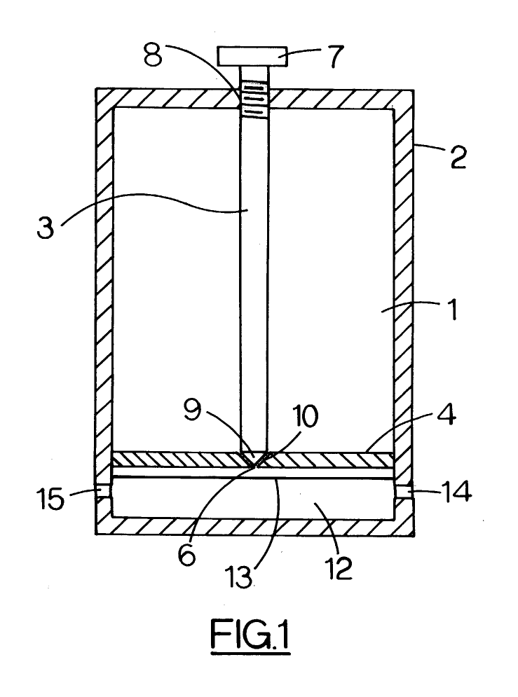

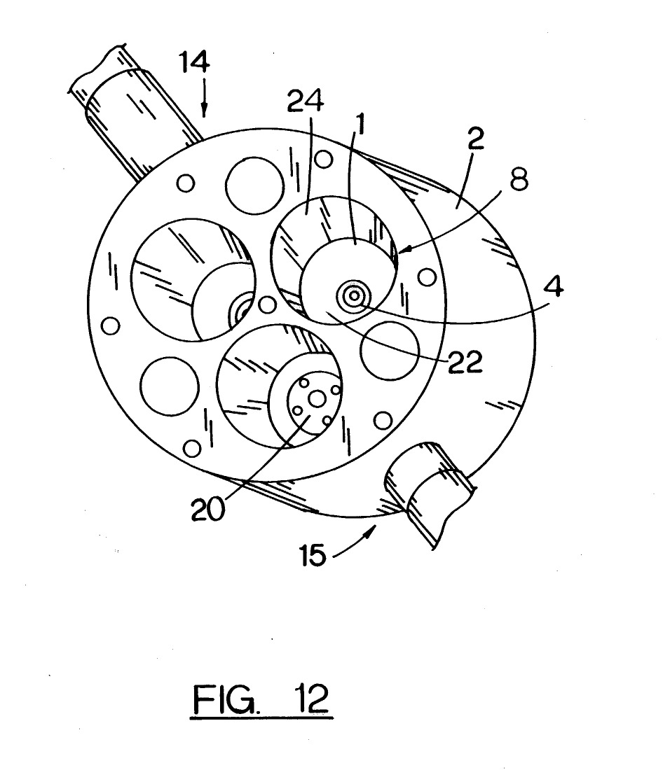

The present invention relates to a portable anesthetic apparatus for evaporating a liquid anesthetic agent into a stream of a carrier gas. As shown in FIG. 1, the liquid anesthetic agent may be contained in a reservoir 1. By incorporating two or more reservoirs 1 into the apparatus as shown in FIG. 12, different liquid anesthetic agents may be contained in the apparatus at one time. Also, a mixture of different liquid anesthetic agents may be administered simultaneously.

Reservoir 1 may be defined by a cylinder 2 made from a material resistant to a liquid anesthetic agent and that is sufficiently rigid to maintain integrity of the reservoir. Although cylinder shapes are preferred, other shapes which form a cavity may be employed. Preferred materials for the cylinder 2 include metals and metal alloys. An especially preferred metal is aluminum.

The cylinders 2 may be individual units dropped into a housing for the portable anesthetic apparatus. However, in a preferred embodiment, the cylinder 2 or cylinders 2 are formed by boring a single piece of metal stock. Thus, the single piece of metal stock may have one, two, three or more cylinders 2 bored into it. In this single stock embodiment, exceptional strength and durability are achieved. This strength and durability are especially beneficial in veterinary medicine or in military applications in which the portable anesthetic apparatus is transferred into the field or operates under hostile conditions such as in the case of frightened livestock or during military action.

A control shaft 3 extends from a top end of the cylinder 1 to a seat of the cylinder 4. As shown in FIG. 1, the control shaft 3 enters an opening in the top end of the cylinder 2 and extends most of the entire vertical length of the cylinder 2 to the seat of the cylinder 4. The control shaft 3 is rotated to control a flow of the drops of the liquid anesthetic agent out of an exit port 6. that is integral to the seat of the cylinder 4. The control shaft 3 removably covers the exit port 6. The control shaft 3 may be rotated by manually rotating a control shaft knob 7. The control shaft 3 enters the interior of the cylinder 2 via the cylinder opening 8. The cylinder opening 8 may contain a seal or ring to maintain the liquid anesthetic agent in the reservoir 1. The seal or ring should be resistant to the liquid anesthetic agent such that that seal or ring does not deteriorate. In a preferred embodiment, the cylinder opening 8 and the control shaft 3 are tapped to be threadably connected.

The control shaft 3 has a tapered portion 9 which is received by a tapered receiving portion 10 of the seat of the cylinder 4. The tapered receiving portion 10 defines an exit port 6 in the seat of the cylinder 4. The exit port 6 has a diameter of about 0.125 inches tapering down to about 0.050 inches.

In operation of the portable anesthetic apparatus of the present invention, drops of the liquid anesthetic agent are released through exit port 6 by rotating the control shaft 3 to allow the liquid anesthetic agent from the reservoir 1 to flow through the exit port 6 of the seat of the cylinder 4. As the control shaft 3 is rotated to an open position, the tapered portion of the control shaft 9 is distanced from the exit port 6 and thus the liquid anesthetic agent is released from reservoir 1.

The tapered portion of the control shaft 9 has an end diameter of about 0.125 inches to about 0.050 inches. The tapered portion of the control shaft 9 may be made from the same piece of stock metal that forms the control shaft 3. Alternatively, the tapered portion of control shaft 3 may be made separately from a different or the same material and then fused to the control shaft 3 by welding or other techniques well known in the art. When not in operation, the tapered portion of the control shaft 9 may be tightened against the tapered receiving portion 10 by rotating the control shaft 3. It is preferred that the control shaft 3, the control shaft knob 7, and the tapered portion of the control shaft 9 are made from a durable material that is resistant to the liquid anesthetic agents and may be formed with precision. A particularly preferred metal is brass. Exit port 6 may be formed by drilling a hole in the seat of the cylinder 4.

In operation, the liquid anesthetic agent is released from the reservoir 1 via the exit port 6 in a drop wise fashion by rotating the control shaft 3. The drops of the liquid anesthetic agent are released into the evaporation chamber 12. The evaporation chamber 12 may also be formed by boring out the single piece of metal stock. The evaporation chamber 12 contains a conductive material 13 of metal shavings, metallic particles, and/or metal mesh sheet which will conduct ambient heat. As the liquid anesthetic agent is released drop wise onto the conductive material 13, the liquid anesthetic agent evaporates into the stream of carrier gas by the heat conducted from the ambient environment through the conductive material 13.

The carrier gas enters the evaporation chamber 12 via a tube or other conduit connected to the evaporation chamber opening 14. As the carrier gas enters the evaporation chamber 12, the liquid anesthetic agent evaporates into the carrier gas to form a mixture of carrier gas and anesthetic agent. The mixture of carrier gas and anesthetic agent exits the evaporation chamber 12 via the evaporation chamber exit 15 through a tube or conduit. From the evaporation chamber exit 15, the mixture of carrier gas and anesthetic agent travels through a tube or conduit to a patient for administration. The flow of the mixture of carrier gas and anesthetic agent to the patient is controlled by the carrier gas entering the evaporation chamber 12.

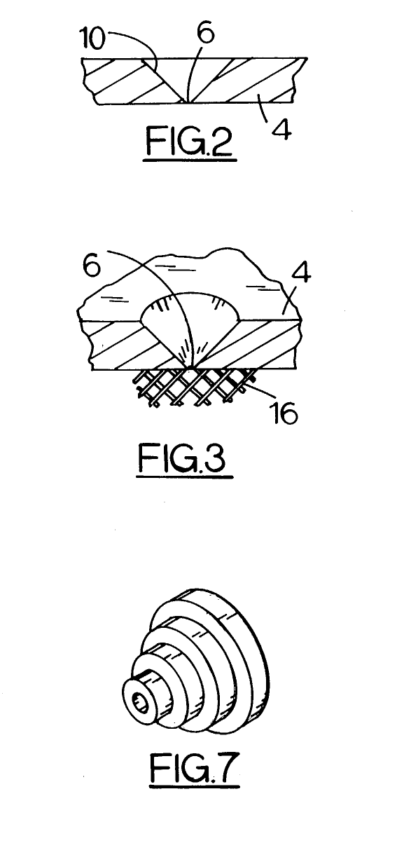

As shown in FIG. 2, the tapered receiving portion 10 may be partially or entirely threaded. In this embodiment, the tapered portion of the control shaft 9 may likewise be partially or entirely threaded. The tapered receiving portion 10 may also have a seal or ring of an elastomeric material to provide positive sealing when the control shaft 3 and the tapered portion of the control shaft 9 are rotated tightly against the tapered receiving portion 10 in a closed position. Preferably, the seal or ring is chemically resistant to the liquid anesthetic agent. It is also possible, to maintain proper sealing, i.e., little or no leakage, with only the contact of the tapered portion of the control shaft 9 against the tapered receiving portion 10.

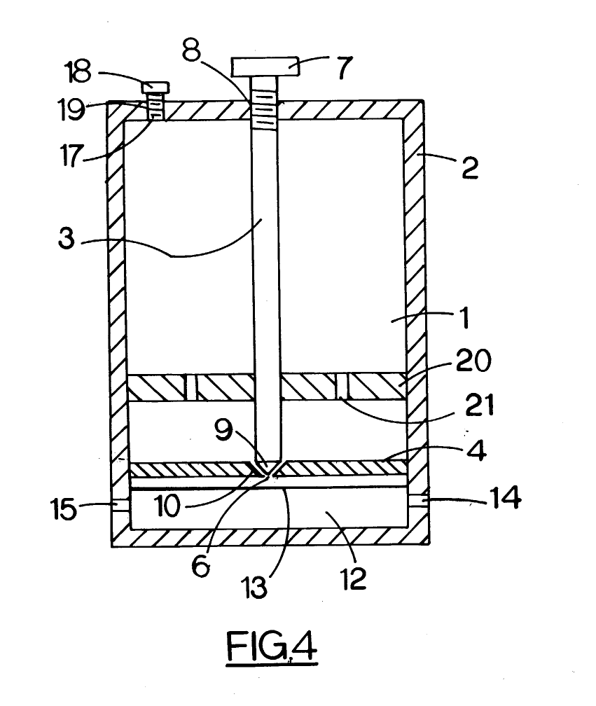

In a preferred embodiment of the present invention, as shown in FIG. 3, a mesh sheet 16 is the conductive material and may be attached below the seat of the cylinder 4 and immediately under the exit port 6. The mesh sheet 16 may be about 1 to about 8 millimeters from the exit port 6. Preferably, the exit port 6 is about 3 to about 5 millimeters from the exit port. In this embodiment, the mesh sheet 16 may be used with or without a conductive material 13. The mesh sheet 16 may be made from stainless steel. The mesh sheet 16 has openings of about 0.010 millimeters to about 0.020 millimeters in diameter. In an especially preferred embodiment, the mesh sheet 16 is used without conductive material 13 and is immediately below the exit port 6 such that the distance between the exit port 6 and the mesh sheet 16 is small enough such that a drop exiting the exit port 6 is not fully formed and is immediately received from the exit port 6 without forming a drop that is suspended in the air and falls to the mesh sheet. The close proximity between the exit port 6 and the mesh sheet 16 provides for the dispersion of the drop of the liquid anesthetic agent over the mesh sheet with reduced “flow through” of the liquid anesthetic agent. If the drop is allowed to fully form and fall on the mesh sheet, some of the drop may pass through the sheet and pool beneath the sheet.

For proper operation of the portable anesthetic apparatus of the present invention, the reservoir 1 must be vented such that a vacuum is not created in the reservoir 1. The venting may occur through the cylindrical opening 8 in the top of the cylinder 2 shown in FIG. 1 through the space between the control shaft 3 and the top of the cylinder 2. The venting may also occur via a gap or perforation in a seal around the cylinder opening 8. The venting may also occur through the threads of an embodiment having a threaded cylinder opening 8.

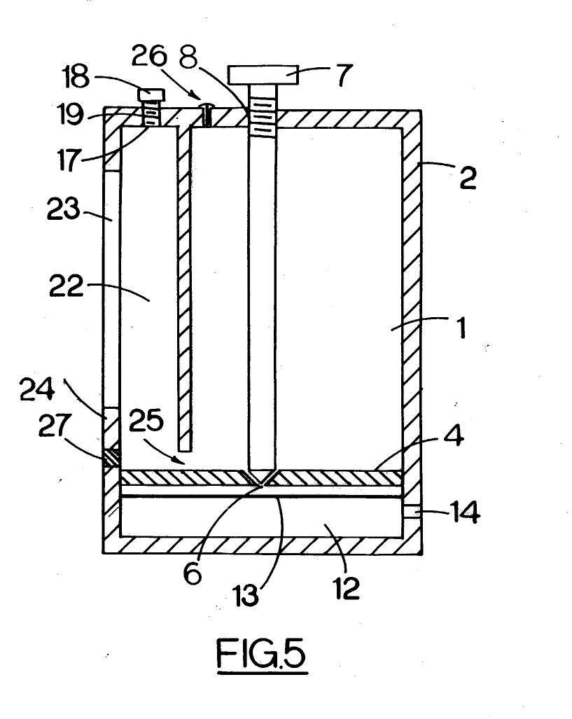

In an embodiment of the present invention shown in FIG. 4, the cylinder 2 has a fill port 17 and a fill cap 18. The fill cap 18 may be removed for filling the reservoir 1 of the cylinder 2 with liquid anesthetic agent. The fill port 17 may also have a fill port vent 19 that is opened by loosening the fill cap 18 which is threadably connected to the top of the cylinder 2. In this embodiment, venting may be accomplished by unscrewing or partially unscrewing the fill cap 18.

Also shown in FIG. 4 are optional axial supports 20. One or more axial supports 20 may be employed to help stabilize the control shaft 3. The axial supports 20 have one or more fluidic channels 21 to provide for the fluidic communication of the liquid anesthetic agent throughout the entirety of reservoir 1.

another embodiment of the present invention as shown in FIG. 5, a fill reservoir 22 may be immediately adjacent to reservoir 1. In this embodiment, the fill reservoir 22 provides visual verification of the level of the liquid anesthetic agent in the reservoir 1 via a borosilicate sight glass 23 which is covering a bored out channel into a fill reservoir wall 24. In this embodiment, the fill reservoir 22 is filled with liquid anesthetic agent via the fill port 17. As the liquid anesthetic agent enters the fill reservoir 22 it moves to the reservoir 1 via the reservoir channel 25 connecting the fill reservoir 22 and the reservoir 1. Thus, the levels of liquid anesthetic agent in both the fill reservoir 22 and the reservoir I will equilibrate. In this embodiment, the fill reservoir 22 is vented by the loosening the fill cap 18 such that the fill port vent 19 may vent. In this embodiment, the reservoir 1 is vented via a one way vent 26.

The embodiment of FIG. 5 may be formed by first boring the reservoir 1 and the fill reservoir 22. Next, the reservoir channel 25 may be bored. Then, a portion of the fill reservoir wall 24 may be filled in with a filler material 27 to fill in the bored portion of the reservoir channel 25 nearest the exterior of the reservoir wall 24. Finally, the channel may be bored in fill reservoir wall 24 and the borosilicate sight glass 23 is placed over the channel. An elastomeric seal, resistant to the liquid anesthetic agent, may be placed between the channel in fill reservoir wall 24 and the borosilicate sight glass 23.

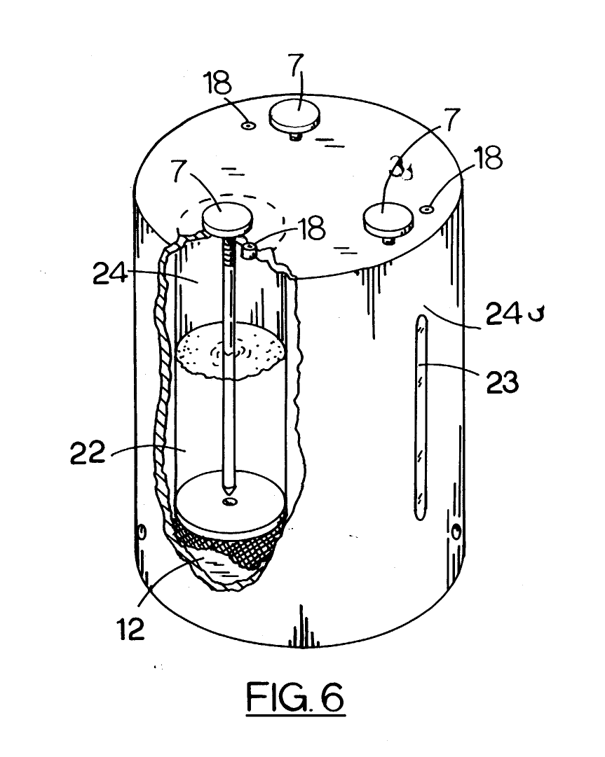

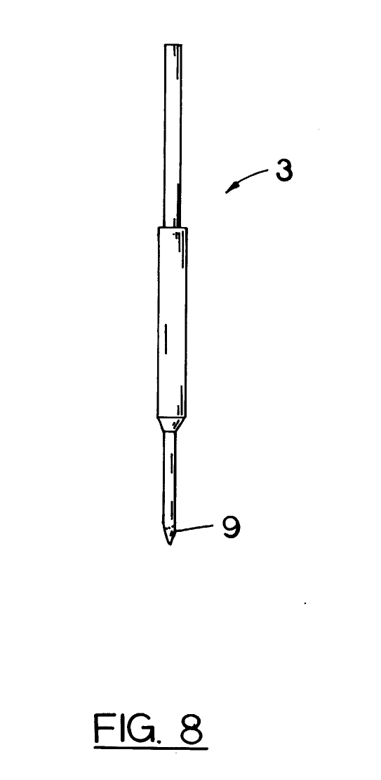

The present invention will now be described in another preferred embodiment shown in FIG. 6. In this embodiment, a three chambered portable anesthetic apparatus is shown. Control shaft knobs 7 and fill caps 18 are shown for each of the three reservoirs 1 and three fill reservoirs 22. In this preferred embodiment with three chambers, the reservoirs 1 and the fill reservoirs 22 and the evaporation chambers 12 are formed by boring a single piece of aluminum stock. This particular anesthetic apparatus made from essentially a single piece of this metal stock provides significant increase in durability. In other embodiments of the present invention, the three cylinders may be formed independently and dropped into a housing.

The fill cap 18 may also have declinations and/or witness lines thereon for accurate measurements. Further, a measuring extension may protrude from the fill cap 18. The top surface of the cylinder 2 may likewise have declinations and/or witness lines thereon.

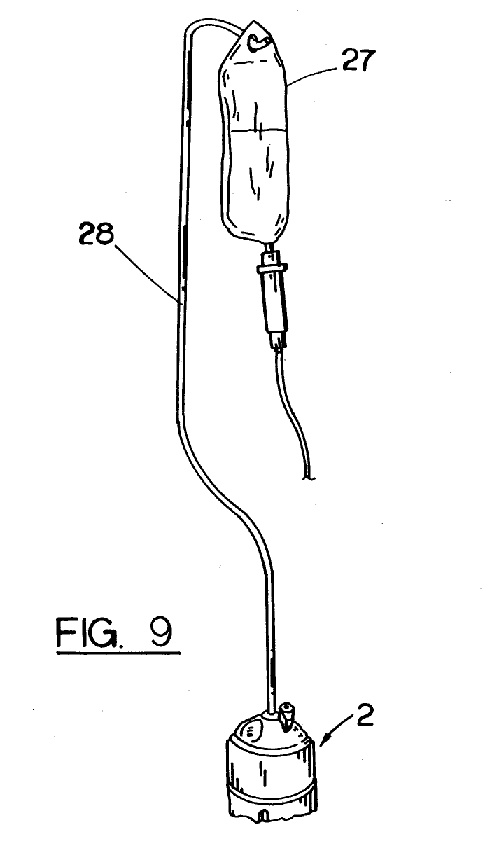

In another embodiment of the present invention, a flexible fluid container such as a VIAFLEX® container by The Baxter Company may be used to supply the fill port 17 with liquid anesthetic agent. As shown in FIG. 9, a flexible fluid container 27 is held by an extension 28. The use of the flexible fluid container may provide for an essentially endless supply of liquid anesthetic agent thus prolonging the use of the apparatus. The flexible fluid container may be made from PVC or other similar flexible material that is resistant to the liquid anesthic agent. In another embodiment of the present invention, the evaporation chamber 12 may have a window of borosilicate sight glass such that the practitioner may observe the interior of the vaporation chamber 12 and conductive material 13 to insure that the liquid anesthetic agent is not collecting or pooling. For accurate operation of the anesthetic apparatus of the present invention, it is preferred that the liquid anesthetic agent is evaporated as the drops enter the evaporation chamber 12 and contact the conductive material 13 and/or mesh sheet 16.

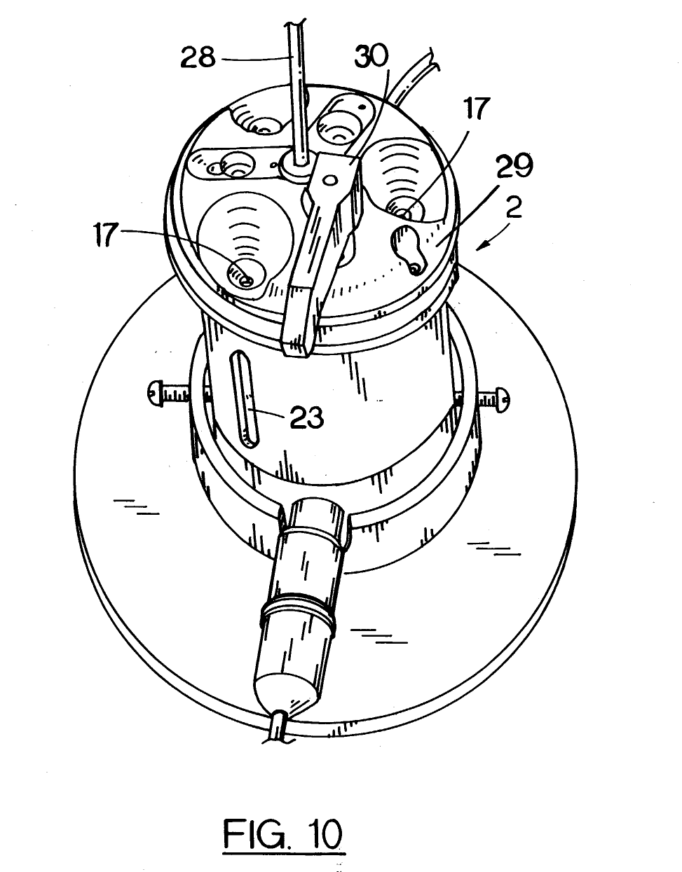

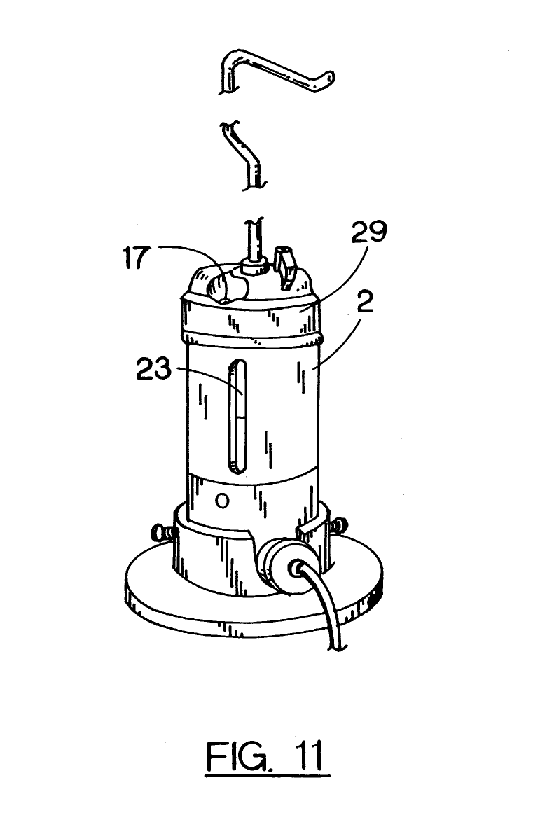

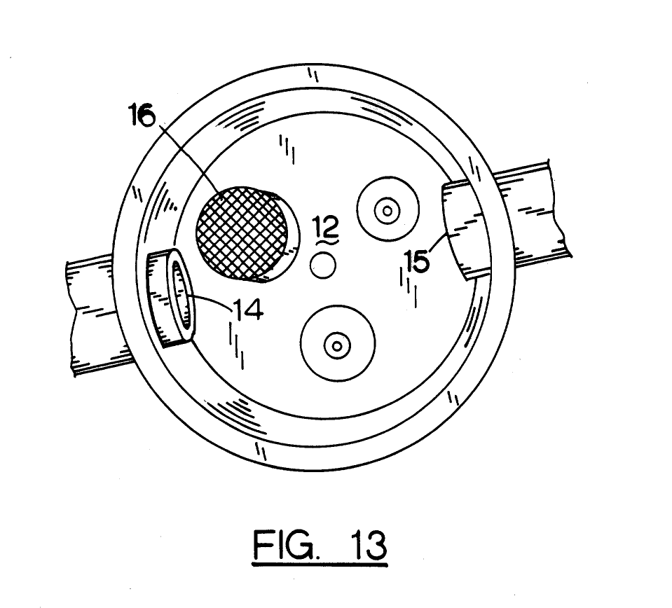

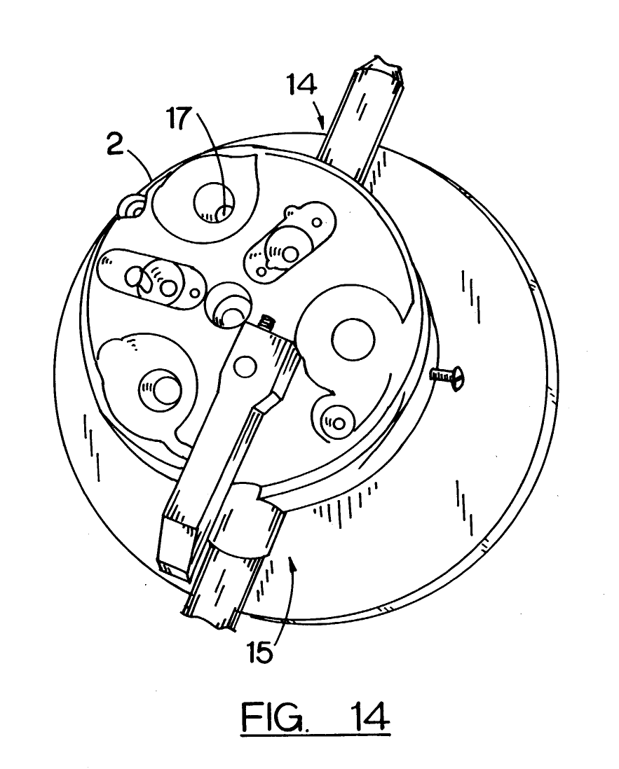

In a preferred embodiment shown in FIG. 10, a top cap 29 is placed over the cylinder 2 to seal the reservoir I or reservoirs 1. The top cap 29 may have declinations and/or witness lines thereon for accurate measurements. Further, a measuring extension 30 may replace or protrude from the control shaft knob 7. In this embodiment, a bottom cap is also placed on the evaporation chamber 12 to seal the evaporation chamber 12. FIG. 12 depicts this embodiment with the top cap 29 removed. FIG. 13 depicts this embodiment with the bottom cap removed to reveal the evaporation chamber 12.

Preferred liquid anesthetic agents for use with the present invention may include Isoflurane, Hallothane, Sevofulorane, and Ethrane.

The portable anesthesia apparatus of the present invention may also include a universal connector as shown in FIG. 7 for connecting carrier gas tubing to the evaporation chamber opening 14 and the evaporation chamber exit 15. The universal connector has a series of openings that are graduated in size. The universal connector may provide connectivity for 16 mm, 19 mm, and 22 mm tubing which are commonly used in anesthetic applications.

The carrier gas used with the portable anesthesia apparatus of the present invention may include carbon dioxide, nitrogen, ambient air, or other inert gases. In the event that there is no source of pressurized gas, the practitioner operating the apparatus of the present invention may provide the carrier gas by the manual operation of a bag valve device for respiration, such as the commercially available AMBU® respiration bag. Thus, the present invention is highly portable and requires no external electricity or dedicated source of carrier gas or even a bulky tank containing the carrier gas. Of course, pressurized carrier gas may be readily available and used accordingly when the present invention is used in a medical facility.

The portable anesthesia apparatus of the present invention may be operated with a carrier gas flow rate of 1-9 liters/minute. For most mammals, anesthetics are administered at a flow rate of about 3 liters/minute with an anesthetic concentration of about 1% to about 6%. Tests of the apparatus show that a drop rate of about 2 drops/second will provide about a 3% concentration of anesthetic at 3 liters/minute.

In the present invention, about 80 to about 130 drops of liquid anesthetic agent equal one milliliter of liquid anesthetic agent. Preferably, about 100 to about 110 drops of liquid anesthetic agent equals one milliliter of liquid anesthetic agent. In a most preferred embodiment, about 106 drops of liquid anesthetic agent equals one milliliter of liquid anesthetic agent.

The conductive material 13 may include aluminum, copper or other conductive metal. The conductive metal conducts the ambient heat from the environment. Heat is required to evaporate the liquid anesthetic agent into the carrier gas.

Depending on the value of the patient and/or the conditions under which anesthesia is performed, the practitioner may use the present invention in conjunction with known in the art gas analysis monitors, physiological monitors, and systems for carbon dioxide removal. The present invention may be incorporated into a closed circuit anesthesia system. The present invention provides a skilled practitioner with an economical and highly accurate mechanical device to administer anesthesia to patients. For the most accurate result, the present invention should be used with the reservoir being of 25-100% full of the liquid anesthetic agent. In this range of 25-100%, pressure applied by the liquid anesthetic agent in the reservoir onto the exit port 6 is relatively constant.

The portable anesthetic apparatus of the present invention provides a practitioner the ability to charge the apparatus with two or more different liquid anesthetic agents and tightly seal the agents in the apparatus for transfer to a remote location. During the administration of the liquid anesthetic agent, the practitioner may administer two or more liquid anesthetic agents simultaneously, serially, or in an overlapping fashion. This flexibility provides a significant advantage over other prior art portable anesthesia apparatus.

What is claimed is:

1. A portable anesthesia apparatus, comprising:
a reservoir defined by walls for containing a liquid anesthetic agent,
a control shaft having a tapered portion,
a seat having a tapered receiving portion,
the seat having an exit port that opens to an evaporation chamber,
wherein the tapered receiving portion of the seat receives the tapered portion of the control shaft,
wherein the exit port is below the tapered receiving portion of the seat, and
wherein a conductive material is below the exit port and contained in the evaporation chamber.

2. The anesthesia apparatus according to claim 1, wherein rotating the control shaft releases the liquid anesthetic agent from the reservoir onto the conductive material.

3. The anesthesia apparatus according to claim 2, wherein the liquid anesthetic agent passes through the exit port prior to contacting the conductive material.

4. The anesthesia apparatus according to claim 1, wherein the conductive material is a mesh sheet.

5. The anesthesia apparatus according to claim 1, wherein the tapered receiving portion and the tapered portion of the control shaft are threadably connected.

6. The anesthesia apparatus according to claim 1, wherein the apparatus comprises three reservoirs defined by walls.

7. The anesthesia apparatus according to claim 1 wherein the apparatus comprises two or more reservoirs defined by walls.

8. The anesthesia apparatus according to claim 1, further comprising a fill reservoir in fluidic communication to the reservoir.

9. The anesthesia apparatus according to claim 1, wherein the walls and the evaporation chamber are formed from a single piece of metal.

10. The anesthesia apparatus according to claim 6, wherein the walls for the three reservoirs are made from a single piece of metal stock.

11. The anesthesia apparatus according to claim 4, wherein the mesh sheet is about 1 to about 8 millimeters from the exit port.

12. The anesthesia apparatus according to claim 2, wherein the liquid anesthetic agent is released from the reservoir in drops of liquid anesthetic agent.

13. The anesthesia apparatus according to claim 12, wherein the conductive material is a mesh sheet, and wherein the mesh sheet is about 1 to about 8 millimeters from the exit port.

14. The anesthesia apparatus according to claim 1, wherein the walls are cylindrical.

15. The anesthesia apparatus according to claim 4, wherein the mesh sheet has openings about 0.010 millimeters to about 0.020 millimeters in diameter.

16. A method of manufacturing an anesthesia apparatus, comprising:
boring a reservoir in a top side of a piece of metal stock, wherein the reservoir defines walls for containing a liquid anesthetic agent;
boring an evaporation chamber in a bottom side of the metal stock and thus forming a seat of the metal stock that separates the evaporation chamber from the reservoir;
drilling an exit port in the seat that provides communication between the evaporation chamber and the reservoir;
inserting a control shaft through the reservoir that is received in the seat, whereby the control shaft removably covers the exit port; and
inserting a conductive material into the evaporation chamber below the exit port.
